(19) United States
(12) Patent Application Publication
(10) Pub. No.: US 2021/0220461 A1
(43) Pub. Date: Jul. 22, 2021

(54) Multivalent Streptococcus Vaccines

(71) Applicant: Inventprise, LLC, Redmond, WA (US)

(72) Inventors: Subhash V. Kapre, Redmond, WA (US); Anup K. Datta, Renton, WA (US); Keith P. Klugman, Seattle, WA (US)

(73) Assignee: Inventprise, LLC, Redmond, WA (US)

(21) Appl. No.: 17/151,445

(22) Filed: Jan. 18, 2021

Related U.S. Application Data
(60) Provisional application No. 62/962,535, filed on Jan. 17, 2020.

(51) Int. Cl. A61K 39/09

(52) U.S. Cl. CPC A61K 39/092; A61K 2039/70

(57) ABSTRACT

The invention is directed to immunogenic compositions, including vaccines, containing multivalent immunogenic composition comprising 25 different serotypes of capsular polysaccharides of S. pneumoniae. Compositions are preferably liquid and thermo stable for periods of time that allow for distribution and use. The invention is also directed to method for the manufacture and methods for the administration of 25 valent immunogenic compositions of S. pneumoniae.

### REFERENCE TO RELATED APPLICATIONS

This application claims priority to U.S. Provisional Application No. 62/962,535, filed Jan. 17, 2020, the entirety of which is incorporated by reference.

### BACKGROUND

### 1. Field of the Invention

The present invention is directed to complexes comprising multivalent compounds, immunogenic compositions, and vaccines comprising carrier protein coupled to bacterial capsular polysaccharides and uses thereof. In particular, compositions of the invention comprise multivalent immunogenic compositions, wherein the bacterial capsular polysaccharides are derived from multiple serotypes of Streptococcus pneumoniae. The carrier protein is coupled to bacterial capsular polysaccharides, through linkers, preferably of defined lengths.

### DESCRIPTION OF THE BACKGROUND

Streptococcus pneumoniae is a Gram-positive pathogen responsible for invasive pneumococcal diseases (IPDs) such as pneumonia, bacteremia, meningitis, and acute Otitis media. Pneumonia is the most common manifestation of invasive pneumococcal disease, whereas bacterial spread within the respiratory tract may result in middle-ear infection, sinusitis or recurrent bronchitis. Pneumococcus is encapsulated with a chemically linked polysaccharide which results in serotype specificity. At least 90 pneumococcal serotypes are known of which about 23 account for 90% of invasive diseases and capsular polysaccharide is a poor immunogen.

There are currently three pneumococcal conjugate vaccines (PCV) available on the global market: PREVNAR®, SYNFLORIX®, and PREVNAR-13®. There is a need to address remaining unmet medical need for coverage of pneumococcal disease due to serotypes not found in PREVNAR-13® and potential for serotype replacement over time. here is a need for immunogenic compositions covering pathogenic serotypes and methodology that can be used to induce a uniform and high immune response against all serotypes including the additional Streptococcus pneumoniae serotypes in humans and in children less than two years old.

A capsular polysaccharide (CPS) is a key virulence determinant and generally insufficiently immunogenic to induce a T cell-dependent immune response in infants and children. Conjugation of a carrier protein to CPS can induce an immune response that undergoes class switching. Accordingly, a 7-valent (PCV-7, Pfizer Inc., USA), a 10-valent (Synflorox-10, GSK Vaccines) and a 13-valent pneumococcal conjugate vaccine (PCV-13, Pfizer Inc., USA) have been developed to efficiently prevent the incidence of IPDs. Reductive amination chemistry and cyanylation chemistry has been widely used to prepare the conjugate vaccines.

U.S. Pat. No. 9,492,559 discloses immunogenic compositions comprising conjugated capsular polysaccharide antigens and uses thereof. The immunogenic compositions disclosed include an 8, 9, 10, 11, 12, 13, 14, 15, 16, 17, 18, 19 or 20-valent pneumococcal conjugate composition. Also disclosed is a 7, 8, 9, 10, 11, 12, 13, 14, 15, 16, 17, 18, 19, 20, 21, 22, 23, 24 or 25-valent pneumococcal conjugate composition.

International Application Publication No. WO 2014/097099A2 discloses a glycol-conjugation process directed to several serotypes in addition to Prevnar-13 valent conjugates. New polysaccharide conjugates are added to formulation to increase efficacy of the vaccine.

U.S. Patent Application Publication No. 2011/023526 discloses a 15-valent pneumococcal polysaccharide-protein conjugate vaccine composition. This patent is directed to 15-valent conjugate vaccines made by adding two or more serotypes with currently available 1-3 vaccines.

International Application Publication No. WO 2016/207905 discloses multivalent pneumococcal conjugate vaccine. This application is directed to a 13 or greater valent conjugate vaccine and deletion of serotype 6A.

U.S. Patent Application Publication No. 2017/007713 discloses a linker containing ((2-oxoethyl) thio) with enhanced functionality.

International Application Publication No. WO 2014/092377 discloses a 13 valent composition wherein 12 serotypes were selected from the group consisting of serotypes 1, 3, 4, 5, 6A, 6B, 7F, 9V, 14, 18C, 19A, 19F, and 23F and one from 12 or 9N.

International Application Publication No. WO 2014/092378 discloses an immunogenic composition having 13 different polysaccharide-protein conjugates wherein each conjugate contained a capsular polysaccharide isolated from 12 serotypes selected from the group consisting of serotypes 1, 3, 4, 5, 6A, 6B, 7F, 9V, 14, 18C, 19A, 19F and 23F, and serotypes 22F or 33F.

Chinese Application Publication No. 101590224 discloses a 14-valent pneumococcal polysaccharide-protein conjugate vaccine containing serotypes 1, 2, 4, 5, 6A, 6B, 7F, 9N, 9V, 14, 18C, 19A, 19F and 23F.

Chinese Application Publication No. 104069488 discloses 14 valent polysaccharide protein conjugate wherein the 14 serotypes were 1, 4, 5, 6A, 6B, 7F, 9V, 14, 18C, 19A, 19F, 22F, 23F and 33F.

International Application Publication No. WO 2016207905 discloses a multivalent pneumococcal conjugate vaccine comprising conjugates of CRM197 and at least 14 capsular polysaccharides selected from serotypes 1, 3, 4, 5, 6B, 7F, 9N, 9V, 14, 15B, 18C, 19A, 19F, 22F, 23F and 33F. U.S. Pat. No. 8,192,746 disclosed a 15 valent immunogenic composition comprising capsular polysaccharides from serotypes 1, 3, 4, 5, 6A, 6B, 7F, 9V, 14, 18C, 19A, 19F, 22F, 23F, and 33F conjugated to CRM197.

International Application Publication No. WO 2013/191459 discloses a 15 valent composition comprising S. pneumoniae capsular polysaccharides form serotypes of 1, 2, 3, 4, 5, 6A, 6B, 7F, 9N, 9V, 14, 18C, 19A, 19F and 23F.

Chinese Application Publication No. 103656632 discloses multi valent pneumococcal capsular polysaccharide composition containing serotype 6A and at least one extra serotype selected from the group consisting of 1, 2, 3, 4, 5, 6B, 7F, 8, 9N, 10A, 11A, 12F, 14, 15B, 17F, 18C, 19A, 19F, 20, 22F, 23F and 33F which provided protection against 24 different pneumococci serotypes.

Chinese Application Publication No. 103656631 discloses a multivalent pneumococcus capsular polysaccharide-protein conjugate composition comprising capsular polysaccharides of pneumococcus of 24 different serotypes viz. 1, 2, 3, 4, 5, 6A, 6B, 7F, 8, 9N, 9V, 10A, 11A, 12F, 14, 15B, 17F, 18C, 19A, 19F, 20, 22F, 23F and 33F.

U.S. Patent Application Publication No. 2016/0324950 discloses immunogenic polysaccharide-protein conjugates comprising a capsular polysaccharide (CP) from Streptococcus agalactiae, also referred to as group B streptococcus (GBS), and a carrier protein, wherein the CP is selected from the group consisting of serotypes Ia, Ib, II, III, IV, V, VI, VII, VIII, and IX. This was meant for treatment of chronic diabetes mellitus, cancer, heart failure, neurologic, and urologic conditions. The carrier protein capsular polysaccharide conjugates varied.

U.S. Pat. No. 5,360,897 discloses immunogenic conjugate comprising reductive amination product of an intact capsular polymer of the bacterial pathogen S. pneumoniae having at least two carbonyl groups and a bacterial toxin or toxoid, said conjugate comprising a cross-linked conjugate in which there is a direct covalent linkage between the capsular polymer and the toxin or toxoid.

U.S. Pat. No. 7,862,823 describes a multivalent conjugate vaccine composition with at least two different carrier proteins.

U.S. Pat. No. 8,808,708 discloses a 13-valent immunogenic composition consisting of polysaccharide-protein conjugates where serotypes consist of 1, 3, 4, 5, 6A, 6B, 7F, 9V, 14, 18C, 19A, 19F and 23F, and wherein the carrier protein is CRMI97.

U.S. Patent Application Publication No. 2009/0017059 discloses an immunogenic composition where serotypes 19A and 19F were conjugated to different bacterial toxoids.

International Application Publication No. WO 2011/110241 describes pneumococcal conjugate immunogenic compositions or vaccines wherein different conjugation chemistries were used for different components of the immunogenic composition or vaccine. Reductive amination was used for the conjugation of at least one serotype and a conjugation other than reductive amination was used for the conjugation of a different serotypes. The conjugation method selected for different serotypes allowed each serotype to be presented using a conjugation method that allowed the best presentation of the saccharide epitope. Some pneumococcal saccharides conjugated well using reductive amination, whereas other pneumococcal saccharides were conjugated differently to allow the ring structure to remain unbroken and provide better results.

U.S. Pat. No. 7,955,605 discloses a process of making carrier protein polysaccharide conjugate consisting serotype 19A where the activated serotype 19A polysaccharide and carrier protein are suspended in dimethyl sulfoxide (DMSO) to form a conjugate.

U.S. Patent Application Publication No. 2010/0074922 discloses immunogenic composition containing 10 or more serotypes wherein 19F capsular saccharide was conjugated to diphtheria toxoid (DT), serotype 18C capsular saccharide is conjugated to tetanus toxoid and serotypes 1, 4, 5, 6B, 7F, 9V, 14 and 23F capsular saccharides are conjugated to Protein D from Haemophilus influenza.

U.S. Patent Application Publication No. 2010/0239604 discloses a composition comprising multivalent S. pneumoniae capsular saccharide conjugates wherein serotype 19A was conjugated to a first bacterial toxoid and 19F is conjugated to a second bacterial toxoid and 2-9 of the S. pneumoniae capsular saccharides are conjugated to protein D. Apart from increasing the scope of protection by developing vaccines which will offer protection against larger number of serotypes, efforts were focused on developing newer methods of synthesis.

U.S. Pat. No. 7,709,001 describes a method of synthesis of carrier protein conjugate of capsular polysaccharide which consists of 1) reacting purified polysaccharide with a mild acid resulting in size reduction 2) reacting the polysaccharide of step 1 with an oxidizing agent in the presence of bivalent cations resulting in an activated polysaccharide; 3) compounding the activated polysaccharide with a carrier protein 4) reacting activated polysaccharide of step 3 and carrier protein with a reducing agent to form a polysaccharide-carrier protein conjugate; and 5) capping unreacted aldehydes in product of step 4 to yield an immunogenic polysaccharide-carrier protein conjugate.

International Application Publication No. WO 2014/097099 discloses a method of synthesizing a carrier protein conjugate, which involves a) reacting a saccharide with 2,2,6,6-tetramethyl-1-piperidinyloxy (TEMPO) and N-chlorosuccinimide (NCS) in an aqueous solvent to produce an activated saccharide; and b) reacting the activated saccharide with a carrier protein comprising one or more amine groups.

U.S. Patent Application Publication No. 2012/321658 discloses an immunogenic composition wherein serotypes 1, 3, 19A and 19F linked to protein carriers either directly or indirectly through a chemistry other than reductive amination, and one or more different saccharides is/are selected from a second group consisting of serotypes 4, 5, 6A, 6B, 7F, 9V, 14, 18C and 23F which is/are linked to a protein carriers) by reductive amination.

Pneumococcal vaccines are based on 1) pneumococcal polysaccharide vaccine and 2) pneumococcal conjugate vaccines. PNEUMOVAX® marketed by Merck comprises of unconjugated polysaccharides belonging to serotypes 1, 2, 3, 4, 5, 6B, 7F, 8, 9N, 9V, 10A, 11A, 12F, 14, 15B, 17F, 18e, 19F, 19A, 20, 22F, 23F and 33F. Infants and young children respond poorly to most pneumococcal polysaccharides. Immunogenicity of poor immunogens is enhanced by conjugating with carrier proteins. Polysaccharide protein conjugate vaccines are made using capsular polysaccharides linked to protein carriers. The conjugate induces T cell dependent enhanced immune response against the specific serotype.

Conjugates are synthesized using various reagents, such as homo bifunctional, hetero bifunctional linkers of varying lengths. Three pneumococcal conjugate vaccines are available in market, PREVNAR®, SYNFLORIX®, and PREVNAR-13®. PREVNAR® is a heptavalent vaccine that contains the capsular polysaccharides from serotypes 4, 6B, 9Y, 14, 18C, 19F and 23F, each conjugated to a carrier protein designated CRM197. SYNFLORIX® is a deca-valent vaccine from GSK Biologicals that incorporates ten capsular polysaccharides conjugated to protein D from NTHi offering coverage against three additional pneumococcal strains, serotypes 1, 5 and 7F. PREVNAR-13® is a tri-deca-valent vaccine containing 13 capsular polysaccharide prepared from thirteen serotype of Streptococcus pneumoniae (1, 3, 4, 5, 6A, 6B, 7F, 9Y, 14, 18C, 19 A, 19F, and 23F) conjugated to a carrier protein designated CRM197.

The need for a specific serotype depends on the region and antibiotic resistance developed. Thus, U.S. Pat. No. 8,192,746 reports a multivalent immunogenic composition having 15 distinct polysaccharide-protein conjugates. Each conjugate consists of a capsular polysaccharide prepared from serotype of Streptococcus pneumoniae (1, 3, 4, 5, 6A, 6B, 7F, 9\1, 14, 18C, 19A, 19F, 22F, 23F, or 33F) conjugated to a carrier protein CRM197. In certain regions, there is a need for vaccines that induce an immune response against serotype 15B, 15C, and 15A.

With the current methods increasing number of polysaccharide antigens in the multivalent conjugate vaccine formulations, the carrier protein content increases. This increase leads to an increase of immune response to the carrier protein which can cause a systemic overload, which lowers the immune response to the specific serotypes.

Thus, there is a need to develop a pneumococcal vaccine that provides uniform protection against increasing number of serotypes, and, in particular, effective protection when the composition contains increased amounts of carrier protein. In addition to offering protection against increasing numbers of serotypes, there is also a need to develop techniques that reduce carrier protein antibodies in spite of an increase in the number of serotypes.

### SUMMARY OF THE INVENTION

The present invention overcomes the problems and disadvantages associated with current strategies and designs and provides new compositions and methods creating high immune response to multiple serotypes of Streptococcus.

One embodiment of the invention is directed to multivalent immunogenic compositions comprising at least capsular polysaccharides of S. pneumoniae serotypes 1, 2, 3, 4, 5, 6A, 6B, 6C, 6D, 7F, 8, 9V, 9N, 9A, 9B, 10A, 11A, 12F, 14, 15B, 15A, 15C, 17F, 18C, 19A, 19F, 20, 22F, 23F, 24F, 33F and 35B. The multivalent S. pneumoniae immunogenic compositions comprises bacterial capsular polysaccharide coupled to carrier protein. Preferred carrier proteins include, for example, CRM (e.g., purified CRM 197 or recombinantly produced CRM), tetanus toxoid fragments (TTHc), tetanus toxin, tetanus toxin heavy chain proteins, diphtheria toxoid, tetanus toxoid, Pseudomonas exoprotein A, Pseudomonas aeruginosa toxoid, Bordetella pertussis toxoid (PT), Clostridium perfringens toxoid, Escherichia coli heat-labile toxin B subunit, Neisseria meningitidis outer membrane complex (e.g., protein PorB), Hemophilus influenzae protein D, Flagellin Fli C, Horseshoe crab Haemocyanin, RSV virus proteins, adenylate cyclase toxin (ACT), 69 KDa protein and Human Papilloma viral protein antigens or its VLP form, Hepatitis B core antigen or its VLP form or derivatives of HBsAg, and/or and fragments, derivatives, and modifications thereof. Coupling may be direct or through a linker such as, preferably, a PEG linker. Preferably, total carrier protein quantity in the multivalent compounded vaccine is significantly lower than the quantity used in the compositions comprising individual polysaccharides of the same cross-reactive serotypes. Preferably, the immunogenic composition further comprises at least one adjuvant selected from the group consisting of aluminum or an aluminum salt, calcium phosphate, a liposome of monophosphoryl lipid A (MPLA), saponin QS-21, and/or a potent TLR7/8 agonist. Preferably the at least one adjuvant comprises an aluminum adjuvant selected from the group consisting of aluminum phosphate, aluminum sulfate and aluminum hydroxide. Preferably, the immunogenic compositions comprise a therapeutically effective amount of the polysaccharides sufficient to generate a protective immune response in an individual. Preferably, the compositions further contain a pharmacologically acceptable carrier.

Another embodiment of the invention is directed to vaccines comprising immunogenic compositions as described here.

Another embodiment of the invention is directed to methods for the manufacture of multivalent immunogenic compositions and vaccine comprising at least 25 different polysaccharides. Methods of manufacture include PS activation via either oxidation and/or cyanylation chemistry. Preferably PS is oxidized by sodium periodate and introduced with either reactive aldehyde or isothiocyanate (—OCN) groups. Coupling strategies include, for example, short and long linker and/or short and long PS. Coupling of PS to carrier protein may be direct, PS to carrier protein, or indirect through one or more linkers. Preferred linkers include, for example, linkers of polyethylene glycol (PEG). Linkages may be monovalent or multivalent (e.g., bivalent, trivalent, etc.).

Another embodiment of the invention comprises methods of administering multivalent immunogenic compositions and vaccines to an individual for the treatment or prevention of a Streptococcus infection, and preferably infection attributable to Streptococcus pneumoniae. Infections that are treatable with immunogenic compositions include, for example, pneumonia, bacteremia, meningitis, and acute Otitis media. Preferably administration comprises intramuscular injection, intraperitoneal injection, intravenous injection, intranasal, oral or transdermal. Preferably the patient is an infant, a toddler, a child, an adolescent, an adult or a senior. Preferred compositions include immunogenic compositions designed for the treatment and/or prevention of infection of infants, of individuals less than 3 years of age, of individuals less than 5 years of age, of individuals less than 15 years of age, in adults, and in individuals greater than 60 years of age.

Other embodiments and advantages of the invention are set forth in part in the description, which follows, and in part, may be obvious from this description, or may be learned from the practice of the invention.

### DESCRIPTION OF THE INVENTION

Streptococcus pneumoniae is a Gram-positive bacterium which can cause diseases including pneumonia, bacteremia, meningitis, and acute otitis media. The microorganisms are encapsulated with a variety of polysaccharides which produces serotype specificity. At least 90 pneumococcal serotypes are known of which about 23 account for 90% of invasive diseases. The protection against invasive disease is directly related to the ability to generate an antibody response that is specific to a particular capsular polysaccharide associated with the microorganisms of the infection, otherwise referred to as serotype specificity.

A multivalent S. pneumoniae immunogenic composition was surprisingly created comprising polysaccharides of at least 25 different capsular polysaccharide serotypes. The presence of additional serotypes over available vaccines Prevnar-13) is predicted to cover an additional about 21-25% more of invasive pneumococcal diseases (IPDs). The serotypes selected, and those not selected, were determined by the presence or absence of the invasive disease-causing isolates post pneumococcal conjugate vaccine (PCV) introduction as determined globally by region, the likely invasive serotypes in children less than about 5 years of age, the regional needs in low and middle-income countries, and determination of newly emerging serotypes in children less than about 5 years of age since, all prioritization based on frequency in Gavi countries, potential for epidemics and known characterization of polysaccharides. There is also a rational for exclusion of certain known serotypes as the not likely to be invasive serotypes in children less than about 5 years of age, as not likely to meet the regional needs in low and middle-income countries, and as not newly emerging serotypes in children less than about 5 years of age since, as not prioritization based on frequency in Gavi countries, as having little to no potential for epidemics, and as not known to be properly characterized. Important criteria for making the selection of serotypes are listed in Table 1.

TABLE 1
Serotypes listed from highest to lowest frequency

| | | | All | | | | | Antibiotic |
|---|---|---|---|---|---|---|---|---|
| | | | ages | | | More | Less | resistant |
| Number of | IPD <5 | | GAVI | | High | invasive | invasive | common |
| additional | non | IPD <5 | and | | PCV | than | than | emerging |
| serotypes | GAVI | GAVI | non-GAVI | Africa <5 | coverage | 19A | 19A | genotype |
| 1 | 15B/C | 2 | 22F | 8 | 22F | 8 | 6C | 35B |
| 2 | 12F | 12F | 8 | 12F | 12F | 12F | 15A | 15B/C |
| 3 | 8 | 8 | 12F | 35B | 24F | 24F | 15B/C | 15A |
| 4 | 22F | 35B | 6C | 15B/C | 10A | 33F | 16F | 24F |
| 5 | 24F | 23B | 15A | 16F | 23B | 22F | 23B | 9N |
| 6 | 10A | 10A | 9N | 15A | 8 | 38 | 35B | 9L |
| 7 | !5A | 45 | 33F | 10A | 15A | 10A | | 12F |
| 8 | 35B | 20 | 23A | 13 | 11A | | | |
| 9 | 23B | 10F | 10A | 17F | 15B/C | | | |
| 10 | 6C | 24F | 11A | 7C | 9N | | | |
| | replace | | | | | | | |
| | 6A | | | | | | | |
| 11 | 33F | 16F | 24F, | 9N | 38 | | | |
| | | | 15B/C, | | | | | |
| | | | 16F, | | | | | |
| | | | 35B | | | | | |
| Number of | 10 | 1 | | | | | | |
| Additional | | | | | | | | |
| strains | | | | | | | | |

As shown in Table 1, 24 valent with 6C to replace 6A to include 11 serotypes that are bolded—2, 8, 10A, 12F, 15A, 15B/C, 22F, 23B, 24F, 33F, 35B. 16F is believed to be prevalent in Africa, but less invasive. 9N is also prevalent in Africa with a high PCV. Global Pneumococcal Sequence Complex 16 has 65% MDR with 38 in high PCV and invasive. 9N protection from 9V is believed weak. Added in the adult data raises the priority for 9N, 23A and 11A. The remaining strains are very low invasiveness 11A, or relatively rare with little to no AMR or genotype signal 7C, 10F, 13, 17F, 20, 45.

The 25 serotypes identified include: serotypes 1, 2, 3, 4, 5, 6B, 6C, 7F, 8, 9N, 9V, 10A, 12F, 14, 15A, 15B/C, 16F, 18C, 19A, 19F, 22F, 23F, 24F, 33F, and 35B. The rational for inclusion of these serotypes is listed in Table 2.

TABLE 2

| Serotypes | Rationale for Inclusion |
|---|---|
| 1, 3, 4, 5, 6B, | Serotypes most likely to cause invasive disease |
| 7F, 9V, 14, 18C, | globally and included in currently licensed |
| 19A, 19F, 23F | Prevenar 13 |
| 22F, 33F | Serotypes known to invasive and included in the |
| | investigational PCV15 |
| 6C | Due to demonstrated cross-protection between |
| | serotype 6B and 6A, serotype 6B in Prevenar 13, is |
| | substituted with serotype 6C, an increasing cause of |
| | invasive, antimicrobial resistant disease |
| 2 | A highly invasive serotype recently associated with |
| | invasive disease in several LMIC, specifically in |
| | Bangladesh, Guatemala and Israel |
| 8 | Invasive disease in children <5 years of age in post |
| | 2010 era in non-Gavi and Gavi countries |
| 9N | Invasive serotype in children <5 years of age for |
| | which there is not demonstrated cross-protection by |
| | serotype 9V in the currently licensed vaccines |
| 10A | In the most common invasive serotypes in pediatric |
| | and adult populations, both in high income |
| | countries as well as LMICs |
| 12F | A highly invasive serotype known to cause |
| | epidemic disease |
| 15A | Common serotype found in invasive disease in |
| | children <5 years of age especially in non-Gavi |
| | countries |
| 15B/C | Common serotype found in invasive disease in |
| | children <5 years of age especially in non-Gavi |
| | countries |
| 16F | Increasingly cause of invasive disease in children <5 |
| | years of age, in Gavi and non-Gavi countries |
| 24F | Emerging invasive serotype recently described as |
| | the most common cause of meningitis in France, as |
| | well as reports from Argentina, Germany, Peru and |
| | Papua New Guinea |
| 35B | Emerging invasive serotypes |

Preferably the immunogenic composition of the disclosure contains at least 25 different serotypes of bacterial capsular polysaccharides of S. pneumonia wherein the polysaccharides (PS) are conjugated and/or coupled to carrier proteins (e.g., PCV formulations). Preferably the PS are sufficiently purified from the desired serotypes of S. pneumonia and conjugated or otherwise coupled to carrier proteins.

Formulations of multivalent immunogenic compositions can be designed for the treatment of specific populations treatment contain mut

Preferred carrier proteins include, for example, cross reactive materials or CRM (e.g., purified CRM 197 or recombinantly produced CRM), tetanus toxoid fragments (TTHc), tetanus toxin, tetanus toxin heavy chain proteins, diphtheria toxoid, tetanus toxoid, Pseudomonas exoprotein A, Pseudomonas aeruginosa toxoid, Bordetella pertussis toxoid (PT), Clostridium perfringens toxoid, Escherichia coli heat-labile toxin B subunit, Neisseria meningitidis outer membrane complex (e.g., protein PorB), Hemophilus influenzae protein D, Flagellin Fli C, Horseshoe crab Haemocyanin, RSV virus proteins, adenylate cyclase toxin (ACT), 69 KDa protein and Human Papilloma viral protein antigens or its VLP form, Hepatitis B core antigen or its VLP form or derivatives of HBsAg, and/or and fragments, derivatives, and modifications thereof.

Coupling of PS to carrier protein may be direct, PS to carrier protein, or indirect through one or more linkers. Preferred linkers include, for example, linkers of polyethylene glycol (PEG). Linkages may be monovalent or multivalent (e.g., bivalent, trivalent, etc.). Linkers are preferably coupled with polysaccharide and/or carrier proteins by connecting to PEG via two hydrazine functional groups cable of covalently compounding with both carrier protein as well as polysaccharides. This creates a class of covalently compounded PEG products that have the additional effect of PEG on their properties compared to conjugates made by conventional methods. PEG has an additional enhancing effect on the immunogenicity of polysaccharides compared to regular conjugates and a depressing effect on the Immune response of carrier proteins. This allows for an efficient and effective composition with large numbers of serotypes. This surprising and unexpected benefits observed are important considerations in developing immunogenic compounds such as vaccines.

The process of coupling can involve, for example, PS activation via either oxidation or cyanylation chemistry. The PS is oxidized by sodium periodate and introduced with either reactive aldehyde or isothiocyanate (—OCN) groups. Coupling strategies include, for example, short and long linker and/or short and long PS.

Preferably the immunogenic composition is prepared as and maintained as a liquid, although composition may be lyophilized and rehydrated before use. Preferably, the compositions, whether liquid or lyophilized, are suitable for storage at room temperatures for periods of time. Preferred periods include weeks, months and years. Preferably the compositions of the invention are thermo-stable at 30° C. for at least 2 years and at 50° C. for at least 3 months.

Also preferred, compositions comprise from about 0.25 ml to about 1.0 ml per dose, and more preferred are doses of about 0.5 ml. Preferably the immunogenic composition comprises 10 micrograms or less of total polysaccharides and total protein per dose. More preferred, immunogenic compositions comprise 4 micrograms or less of total polysaccharides per dose. Preferably carrier protein comprises from about 0.5% to about 0.7%, by weight, per dose. Also preferred are immunogenic compositions which comprise greater amount by weight of capsular polysaccharides to total carrier protein, although compositions may contain about equal amount by weight of capsular polysaccharides to total carrier protein.

Compositions of the invention may include stabilizers to maintain efficacy for long periods and over multiple temperatures. Stabilizers and protective agents include, for example, excipients, buffers (e.g., citrate, calcium carbonate), amino acids (e.g., lysine, arginine, glycine, etc.), salts, bulking agents, antioxidants and dispersants. Protectant agents include, for example, dextran and other lower molecular weight sugars such as sucrose, trehalose, mannitol, and/or medium-chain triglyceride (MCT) oil.

Protection against pneumococcal disease is obtained by the generation of an immunological response in the individual administered the composition. Suitable immunological response preferably includes the generation of protective antibodies against the different polysaccharide components. Preferably the antibodies observed after administration of the immunological composition fall slowly, more slowly that the rapid reductions observed with convention PCV vaccines. This eliminates a need for multiple injections, so that good protection is achieved after one or at most two injections, saving cost as well as pain to patients such as infants and children (e.g., greater than 3 months). In addition, have a reduced number of injections allows protection to be more widely available than conventional multiple injections, especially for those unable to return for repeated injections.

A preferred formulation of the immunogenic composition comprises at least 25 different serotypes of bacterial capsular polysaccharides of Streptococcus pneumoniae covalently connected to carrier protein through, preferably, PEGylated linker compounds. The carrier protein is covalently connected to bacterial capsular polysaccharides through a number of multifunctional PEG linkers, which may be homo-multi-functional or hetero-multi-functional, and preferably of defined lengths. Preferred linkers include adipic acid di-hydrazide (ADH) and PEGylated-ADH linkers. Preferable, the linkers are from 1 KDa to 3.5 kDa, and may be greater than 3.5 KDa. Preferred hetero- and/or homo-linker sizes include, for example, 40 Å and less, 30 Å and less, 20 Å and less, 10 Å and less, 5 Å and less, 2 Å and less, and/or combinations of these sizes. Preferably the polysaccharide-protein covalent PEG compound is prepared wherein carrier protein reacts with cleaved and depolymerized polysaccharide fragments of optimum chain length. Polysaccharides are conjugated to carrier protein, either of which may be coupled via an activating agent, such as for example, carbodiimide (e.g. 1-ethyl-3(3-dimethylaminopropyl; EDC or EDAC), to give a derivatized carrier protein in presence of a 2-(N-morpholino) ethane sulphonic acid (MES buffer) or EDC/sNHS chemistry. Carbodiimide conjugation, as with CDI-mediated conjugation, works by activating carboxyl groups for direct reaction with primary amines via amide bond formation. EDC couples primary amines to carboxylic acids by creating activated ester leaving groups. Basically, the carbonyl of acid attacks the carbodiimide of EDC creating a proton transfer. The primary amine attacks the carbonyl carbon of the acid forming a tetrahedral intermediate which forms an amine and discards urea.

Preferably the immunological composition contains PS with low molecular weight and bifunctional linkers preferably that enhance immunogenicity. Preferred molecular sizes are 300 kDa and lower, 200 KDa and lower, 100 KDa and lower, 75 Kda and lower, 50 kDa and lower, 25 KDa and lower, 10 KDa and lower, and/or combinations of these molecular sizes. This provides higher immunogenicity and higher avidity of bivalent compounds as well as lower carrier protein immunogenicity.

Another embodiment of the invention is directed to methods for the administration of vaccines of the invention to patients in need thereof for treating or preventing an infection. The method comprises administering a therapeutically effective amount of the vaccine of the invention to a mammal, comprising determining the therapeutically effective amount of the vaccine to be administered that provides therapy to an infected patient and/or protection from infection. The therapeutically effective amount is typically determined by based on the weight of the mammal and the strength or responsiveness of the patient's immune system and can be determined by those skilled in the art. The therapeutically effective amount is administered to a patient in need thereof, which may be to treat an active or suspected infection or prevent an infection. The vaccine may have been obtained from a lyophilized powder and reconstituted to an aqueous or non-aqueous liquid prior to administration to the patient. Preferably the vaccine is administered as a liquid, which may be administer via intra-muscular, intra-peritoneal, or intra-venous injection, and the patient may be an infant, a toddler, an adolescent, an adult or a senior. Surprisingly, the compositions of the invention do not generate side effects such as redness or inflammation at the injection site, and does not generate a generalized fever or inflammation, or other unwanted side effects for the patient. Preferably an immunologically effective vaccine contains only the multivalent composition and nothing further such as, for example, no added adjuvants.

Preferably the immunogenic compositions of the invention comprises administering multivalent immunogenic compositions to an individual for the treatment or prevention of a Streptococcus infection, and preferably infection attributable to Streptococcus pneumoniae. Infections that are treatable with immunogenic compositions include, for example, pneumonia, bacteremia, meningitis, and acute Otitis media. Preferably administration comprises intramuscular injection, intraperitoneal injection, intravenous injection, intranasal, oral or transdermal. Preferably the patient is an infant, a toddler, a child, an adolescent, an adult or a senior. Preferred compositions include immunogenic compositions designed for the treatment and/or prevention of infection of infants, of individuals less than 3 years of age, of individuals less than 5 years of age, of individuals less than 15 years of age, in adults, and in individuals greater than 60 years of age.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. An immunogenic composition comprising at least 25 different serotypes of polysaccharides of S. pneumoniae serotypes coupled to carrier protein.

2. The immunogenic composition of claim 1, wherein one or more of the capsular polysaccharides are from about 10 kDa to about 50 kDa.

3. The immunogenic composition of claim 1, wherein one or more of the capsular polysaccharides from about 30 KDa to about 100 KDa.

4. The immunogenic composition of claim 1, wherein one or more of the capsular polysaccharides are from about 100 KDa to about 300 KDa.

5. The immunogenic composition of claim 1, wherein the carrier protein comprises CRM, purified CRM197, recombinantly produced CRM, tetanus toxoid fragments (TTHc), tetanus toxin, tetanus toxin heavy chain proteins, diphtheria toxoid, tetanus toxoid, Pseudomonas exoprotein A, Pseudomonas aeruginosa toxoid, Bordetella pertussis toxoid (PT), Clostridium perfringens toxoid, Escherichia coli heat-labile toxin B subunit, Neisseria meningitidis outer membrane complex, protein PorB, Hemophilus influenzae protein D, Flagellin Fli C, Horseshoe crab Haemocyanin, RSV virus proteins, adenylate cyclase toxin (ACT), 69 KDa protein and Human Papilloma viral protein antigens or its VLP form, Hepatitis B core antigen or its VLP form or derivatives of HBsAg, and/or and fragments, derivatives, and modifications thereof.

6. The immunogenic composition of claim 1, wherein one or more of the polysaccharides are covalently coupled to a linker and the linker is coupled to the carrier protein.

7. The immunogenic composition of claim 1, which is a liquid.

8. The immunogenic composition of claim 1, which comprises from about 0.25 ml to about 1.0 ml per dose.

9. The immunogenic composition of claim 1, which comprises about 0.5 ml per dose.

10. The immunogenic composition of claim 1, which comprises 10 micrograms or less of total polysaccharides and protein per dose.

11. The immunogenic composition of claim 1, which comprises 4 micrograms or less of total polysaccharides and protein per dose.

12. The immunogenic composition of claim 1, wherein the carrier protein comprise from about 0.5% to about 0.7%, by weight, per dose.

13. The immunogenic composition of claim 1, which comprises about equal amount by weight of capsular polysaccharides to total carrier protein.

14. The immunogenic composition of claim 1, which comprises a greater amount by weight of capsular polysaccharides to total carrier protein.

15. The immunogenic composition of claim 1, further comprising of at least one adjuvant.

16. The immunogenic composition of claim 15, wherein the at least one adjuvant is selected from the group consisting of aluminum salt, calcium phosphate, a liposome of monophosphoryl lipid A (MPLA), saponin QS-21, a TLR7/8 agonist, and combinations thereof.

17. The immunogenic composition of claim 16, wherein the aluminum salt is selected from the group consisting of aluminum phosphate, aluminum sulfate and/or aluminum hydroxide.

18. The immunogenic composition of claim 1, wherein the at least 25 different serotypes of capsular polysaccharides of S. pneumoniae comprise serotypes: 1, 2, 3, 4, 5, 6B, 6C, 7F, 8, 9N, 9V, 10A, 12F, 14, 15A, 15B, 15C, 16F, 18C, 19A, 22F, 23F, 24F, 33F, and 35B.

19. The immunogenic composition of claim 1, which, upon administration to a subject, generates a minimal immune response to carrier protein as compared to the immune response to polysaccharide.

20. The immunogenic composition of claim 1, which provides effective treatment or prevention of infection by S. pneumoniae bacteria.

21. The immunogenic composition of claim 1, comprising a a pharmacologically acceptable carrier.

22. The method for manufacture of the immunogenic composition of claim 1, comprising:
activating carrier proteins to form activated carrier proteins;
reducing a disulfide of each carrier protein to create a sulfhydryl group; and
coupling capsular polysaccharides to the activated carrier proteins.

23. The method of claim 18, wherein the activated carrier proteins are selected from the group consisting of cross-reactive material (CRM197) obtained or derived from C. diptheriae, and recombinant CRM197 obtained or derived from P. fluorescens or E. coli.

24. The method of claim 18, further comprising coupling PEG spacers to the activated carrier proteins.

25. An immunogenic composition comprising one or more polysaccharides of one of more different serotypes of S. pneumoniae coupled to carrier protein via adipic acid dihydrazide (ADH) linkers, wherein the one or more polysaccharides have a molecular weight of from about 100 KDa to about 300 KDa.

26. The immunogenic composition of claim 25, wherein the one or more of the ADH linkers are pegylated dihydrazide (HZ-PEG-HZ) linkers.

27. The immunogenic composition of claim 25, wherein the carrier protein comprises CRM, recombinantly produced CRM, or a domain of CRM.

28. A method of manufacture of the immunogenic composition of claim 25, comprising:
providing the one or more polysaccharides of one of more serotypes of S. pneumoniae and carrier protein;
activating the one or more polysaccharides and/or the carrier protein with pegylated-ADH linkers; and
linking the polysaccharides to carrier protein via carbodiimide chemistry.

29. The method of claim 28, wherein the carrier protein comprises CRM, recombinantly produced CRM, tetanus toxoid or toxoid fragments (TTHc), tetanus toxin, tetanus toxin heavy chain proteins, diphtheria toxoid, tetanus toxoid, Pseudomonas exoprotein A, Pseudomonas aeruginosa toxoid, Bordetella pertussis toxoid (PT), Clostridium perfringens toxoid, or a combination thereof.

30. The method of claim 28, wherein the one or more serotypes comprise serotypes: 1, 2, 3, 4, 5, 6B, 6C, 7F, 8, 9N, 9V, 10A, 12F, 14, 15A, 15B, 15C, 16F, 18C, 19A, 22F, 23F, 24F, 33F, and 35B.
